(19) United States
(12) Patent Application Publication
(10) Pub. No.: US 2014/0187946 A1
(43) Pub. Date: Jul. 3, 2014

(54) ACTIVE ULTRASOUND IMAGING FOR INTERVENTIONAL PROCEDURES

(71) Applicant: GENERAL ELECTRIC COMPANY, Schenectady, NY (US)

(72) Inventors: James Vradenburg Miller, Niskayuna, NY (US); Kedar Anil Patwardhan, Niskayuna, NY (US)

(73) Assignee: GENERAL ELECTRIC COMPANY, Schenectady, NY (US)

(21) Appl. No.: 13/731,213

(22) Filed: Dec. 31, 2012

(51) Int. Cl. A61B 8/08; A61B 8/00

(52) U.S. Cl. CPC A61B 8/5207; A61B 8/0833; A61B 8/4245; A61B 8/463
USPC 600/440; 600/443

(57) ABSTRACT

A computer-implemented method for active control of ultrasound image acquisition includes accessing image data representing a series of ultrasound images acquired over a period of time from an ultrasound probe and identifying an object of interest in at least one of the images. The method further includes detecting changes in a position of the ultrasound probe and/or an orientation of the ultrasound probe over the period of time with respect to the object of interest, or changes in a position of the object of interest and/or an orientation of the object of interest over the period of time with respect to the ultrasound probe. The method further includes adjusting at least one ultrasound image acquisition parameter based on the detected changes in the position and/or the orientation of the ultrasound probe and/or based on the detected changes in the position and/or the orientation of the object of interest.

### FIELD

This disclosure relates generally to medical imaging, and more particularly, to systems and methods of active ultrasound imaging for interventional procedures.

### BACKGROUND

Some conventional ultrasound probes have several adjustable image acquisition parameters, including, for example, spatial resolution, field of view, frame rate, and depth and frequency of the ultrasound signal. These parameters can be adjusted manually by a physician or clinician during an interventional procedure as needed. However, adjusting or changing one image acquisition parameter can affect other image acquisition parameters due to certain performance limitations of the ultrasound probe. For instance, widening the field of view may require decreasing the resolution, while increasing the spatial resolution may require narrowing the field of view.

While performing an interventional ultrasound scanning procedure, initially the user may manually select a wide field of view, at a low resolution, for locating and identifying an object of interest in the patient, and then manually switch to a narrower field of view encompassing the object of interest at a higher resolution. In addition to positioning and orienting the ultrasound probe, the manual switching of parameters involves additional inputs from the user. Thus, it can be difficult to manually adjust various image acquisition parameters, such as spatial resolution, field of view, frame rate, depth and frequency, while simultaneously manipulating the position and orientation of the ultrasound probe.

### SUMMARY

According to one embodiment, a computer includes a processor and a memory operatively coupled to the processor. A computer-implemented method for active control of ultrasound image acquisition using the computer includes accessing, by the processor, image data representing a series of ultrasound images acquired over a period of time from an ultrasound probe operatively coupled to the processor. The method further includes identifying, by the processor, an object of interest in at least one ultrasound image in the series of ultrasound images, and detecting, by the processor, changes in a position of the ultrasound probe and/or an orientation of the ultrasound probe over the period of time with respect to the object of interest, and/or detecting changes in a position of the object of interest and/or an orientation of the object of interest over the period of time with respect to the ultrasound probe. The method further includes adjusting, by the processor, at least one ultrasound image acquisition parameter for acquiring at least one additional ultrasound image using the ultrasound probe based on the detected changes in the position and/or the orientation of the ultrasound probe, and/or at least one ultrasound image acquisition parameter for acquiring at least one additional ultrasound image using the ultrasound probe based on the detected changes in the position and/or the orientation of the object of interest.

In some embodiments, at least one of the ultrasound image acquisition parameters may include a depth of a signal emitted by the ultrasound probe, a frequency of the signal emitted by the ultrasound probe, a spatial resolution of the ultrasound image, a field of view of the ultrasound image, and/or an acquisition frame rate of the ultrasound image.

In some embodiments, the step of adjusting may include increasing or decreasing the spatial resolution such that the object of interest is visible within the at least one additional ultrasound image based on a set of predefined rules, increasing or decreasing the field of view such that the object of interest appears in the at least one additional ultrasound image based on the set of predefined rules, increasing or decreasing the acquisition frame rate based on the set of predefined rules, increasing or decreasing the depth of the signal based on the set of predefined rules, and/or increasing or decreasing the frequency of the signal based on the set of predefined rules.

In some embodiments, the step of adjusting may include automatically steering the field of view based on the detected changes in the position and/or the orientation of the ultrasound probe and/or the object of interest such that the object of interest remains substantially encompassed within the field of view.

In some embodiments, the method may further include simultaneously displaying a wide field of view and a narrow field of view via a user interface operatively coupled to the processor.

In some embodiments, the object of interest may include an anatomical structure in a patient, a surgical instrument inserted into the patient, a device, and/or a marker placed into the patient. In some embodiments, the step of identifying the object of interest may include using one or more image analysis techniques including low level feature detection, statistical model fitting, machine learning, and/or image and model registration. In some embodiments, at least one of the steps of identifying, detecting and adjusting may be further based at least in part on concurrent multimodal input information (e.g., ultrasound and X-ray inputs).

According to one embodiment, a non-transitory computer-readable medium has stored thereon computer-executable instructions that when executed by a computer cause the computer to receive data representing a series of ultrasound images acquired over a period of time from an ultrasound probe operatively coupled to the processor, identify an object of interest in at least one ultrasound image in the series of ultrasound images, detect changes in a position of the ultrasound probe and/or an orientation of the ultrasound probe over the period of time with respect to the object of interest, and/or detect changes in a position of the object of interest and/or an orientation of the object of interest over the period of time with respect to the ultrasound probe, and adjust at least one ultrasound image acquisition parameter for acquiring at least one additional ultrasound image using the ultrasound probe based on the detected changes in the position and/or the orientation of the ultrasound probe, and/or at least one ultrasound image acquisition parameter for acquiring at least one additional ultrasound image using the ultrasound probe based on the detected changes in the position and/or the orientation of the object of interest.

In some embodiments, the computer-readable medium may further include computer-executable instructions that when executed by the computer cause the computer to increase or decrease the spatial resolution such that the object of interest is visible within the at least one additional ultrasound image based on a set of predefined rules, increase or decrease the field of view such that the object of interest appears in the at least one additional ultrasound image based on the set of predefined rules, increase or decrease the acquisition frame rate based on the set of predefined rules, increase or decrease the depth of the signal based on the set of predefined rules, and/or increase or decrease the frequency of the signal based on the set of predefined rules.

In some embodiments, the computer-readable medium may further include computer-executable instructions that when executed by the computer cause the computer to automatically steer the field of view based on the detected changes in the position and/or the orientation of the ultrasound probe and/or the object of interest such that the object of interest remains substantially encompassed within the field of view.

In some embodiments, the computer-readable medium may further include computer-executable instructions that when executed by the computer cause the computer to simultaneously display a wide field of view and a narrow field of view via a user interface operatively coupled to the processor.

In some embodiments, the computer-readable medium may further include computer-executable instructions that when executed by the computer cause the computer to identify the object of interest by using one or more image analysis techniques including low level feature detection, statistical model fitting, machine learning, and image and model registration.

According to one embodiment, a system for active control of ultrasound image acquisition includes a processor, an input operatively coupled to the processor and configured to receive data representing a series of ultrasound images, and a memory operatively coupled to the processor. The memory includes computer-executable instructions that when executed by the processor cause the processor to receive data representing a series of ultrasound images acquired over a period of time from an ultrasound probe operatively coupled to the processor, identify an object of interest in at least one ultrasound image in the series of ultrasound images, detect changes in a position of the ultrasound probe and/or an orientation of the ultrasound probe over the period of time with respect to the object of interest, and/or detect changes in a position of the object of interest and/or an orientation of the object of interest over the period of time with respect to the ultrasound probe, and adjust at least one ultrasound image acquisition parameter for acquiring at least one additional ultrasound image using the ultrasound probe based on the detected changes in the position and/or the orientation of the ultrasound probe, and/or at least one ultrasound image acquisition parameter for acquiring at least one additional ultrasound image using the ultrasound probe based on the detected changes in the position and/or the orientation of the object of interest.

In some embodiments, the memory may further include computer-executable instructions that when executed by the processor cause the processor to increase or decrease the spatial resolution such that the object of interest is visible within the at least one additional ultrasound image based on a set of predefined rules, increase or decrease the field of view such that the object of interest appears in the at least one additional ultrasound image based on the set of predefined rules, increase or decrease the acquisition frame rate based on the set of predefined rules, increase or decrease the depth of the signal based on the set of predefined rules, and/or increase or decrease the frequency of the signal based on the set of predefined rules.

In some embodiments, the memory may further include computer-executable instructions that when executed by the processor cause the processor to automatically steer the field of view based on the detected changes in the position and/or the orientation of the ultrasound probe and/or the object of interest such that the object of interest remains substantially encompassed within the field of view.

In some embodiments, the memory may further include computer-executable instructions that when executed by the computer cause the computer to simultaneously display a wide field of view and a narrow field of view via a user interface operatively coupled to the processor.

### BRIEF DESCRIPTION OF THE DRAWINGS

Features and aspects of embodiments are described below with reference to the accompanying drawings, in which elements are not necessarily depicted to scale.

FIG. 1 is a block diagram of an example of a system for analyzing an ultrasound image, in accordance with an embodiment.

FIG. 2 is a flow diagram of an example of a process for analyzing an ultrasound image, in accordance with an embodiment.

FIG. 3 is a flow diagram of an example of a process for analyzing an ultrasound image, in accordance with an embodiment.

FIGS. 4A and 4B depict examples of a display interface for displaying ultrasound images, in accordance with some embodiments.

FIGS. 5A, 5B and 5C depict examples of various fields of view of ultrasound images, in accordance with some embodiments.

### DETAILED DESCRIPTION

Various embodiments of the present disclosure are directed to active ultrasound imaging for interventional procedures. In some embodiments, one or more ultrasound image acquisition parameters can be automatically controlled based on a context in which the user is using an ultrasonic probe.

According to some embodiments, a computer-implemented image processing method, which may be performed in real-time (e.g., contemporaneously), provides active control of one or more image acquisition parameters during the scan process by detecting an object of interest in the ultrasound image and tracking changes in the position and/or the orientation of the object of interest, or by tracking changes in the position and/or the orientation of the ultrasound probe. Such changes may be indicative of a context in which the user is operating the ultrasound probe. The context can be used as a basis for selecting individual image acquisition parameters or combinations of parameters that provide the most advantageous visualizations within the context. For example, while the user is guiding a surgical instrument into position within a patient, the ultrasound imaging can automatically be switched from a wide field of view for providing a broad anatomical context at a low spatial resolution and frame rate, to a narrow field of view for providing a detailed, high resolution view of the tool at a high frame rate. The former provides the user with a broad anatomical context, while the latter provides the user with a detailed view for precisely manipulating the tool or other device into position. In another example, if the surgical instrument disappears from the narrow field of view, or if the user displaces the position and/or orientation of the ultrasound probe such that the instrument is no longer within the field of view, the ultrasound imaging can automatically be switched back from the narrow field of view to the wide field of view to permit the user to re-locate the instrument in the broad anatomical view.

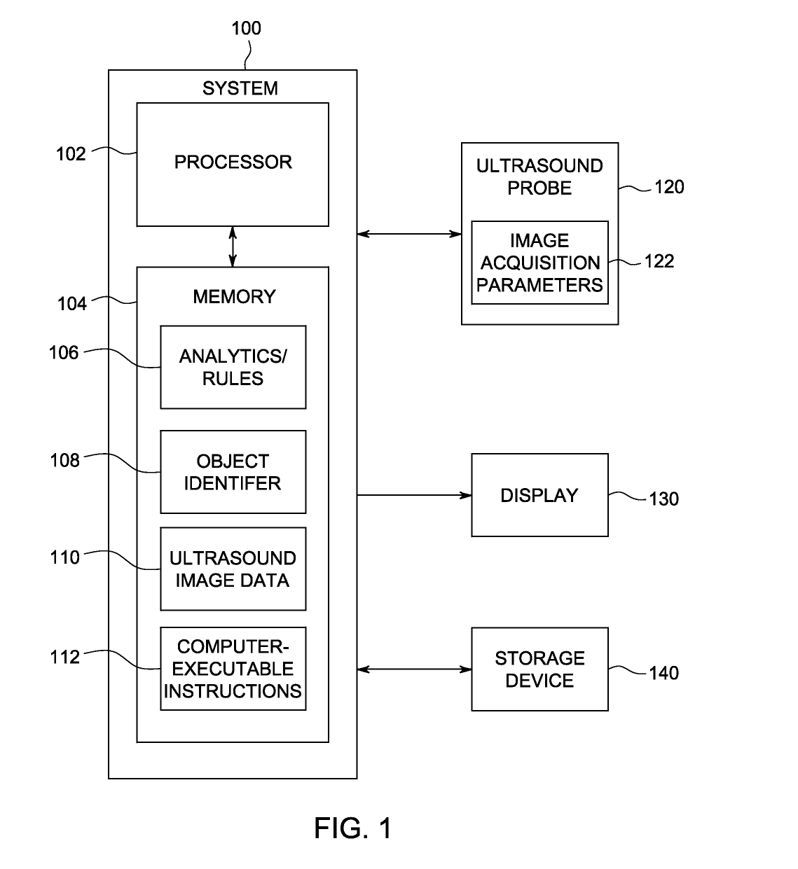

FIG. 1 is a block diagram of an example of a system 100 for analyzing an ultrasound image, according to an embodiment. The system 100 includes a computer having a processor 102 and a memory 104 operatively coupled to the processor 102. The memory is configured to store data representing analytics and/or rules 106 for processing an ultrasound image and an object identifier 108. The memory is further configured to store ultrasound image data 110 representing the ultrasound image, and computer-executable instructions 112 that can be executed by the processor 102 to implement, for example, detection and tracking of an object of interest in the ultrasound image and for displaying the ultrasound image. The system 100 may be operatively coupled to an ultrasound probe 120 for receiving the ultrasound image data 110 therefrom and sending image acquisition parameters 122 thereto. The system 100 may be operatively coupled to a display 130 for displaying ultrasound images. The system 100 may be operatively coupled to a storage device 140 for storing and/or retrieving, for example, data representing image data, training data, statistical model data and/or computer-executable instructions.

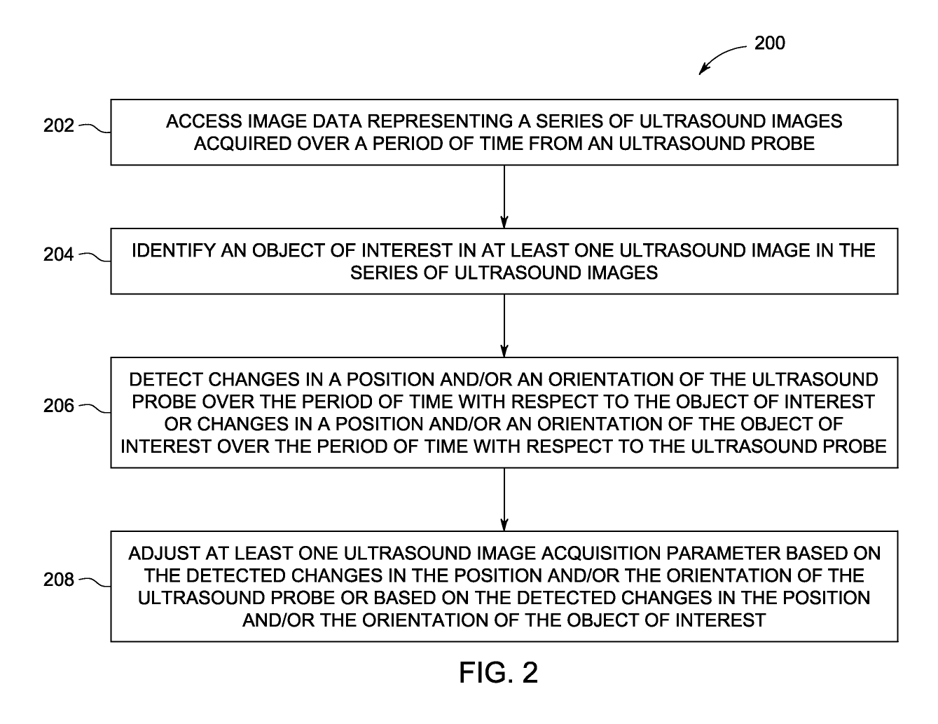

FIG. 2 is a flow diagram of an example of a process 200 for analyzing an ultrasound image, according to an embodiment. At step 202, image data representing a series of ultrasound images acquired over a period of time from an ultrasound probe is accessed. For example, the series of ultrasound images may be generated as an user manipulates the ultrasound probe. The images can be accessed at substantially the same time as the ultrasound probe is acquiring image data (e.g., in real-time or contemporaneously). At step 204, an object of interest in at least one in the series of ultrasound images is identified. The object of interest may include, for example, an anatomical structure in a patient (e.g., an organ, tissue, bone, etc.), a surgical instrument inserted into the patient, a device (e.g., a valve, a pacemaker lead, a cathode ray tube (CRT) lead, a plug, etc.), or a marker placed into the patient. In some embodiments, a portion of a tool, such as the tip, can be the object of interest.

The object identifier 108 of FIG. 1 can be used to identify the object of interest. For example, the object identifier 108 may include a statistical model representing an image of a known object. The object of interest may be identified by comparison to the statistical model. Other techniques known in the art may be utilized, including low level feature detection, statistical model fitting, machine learning, and image and model registration. In some embodiments, the object of interest can be identified based on inputs received from one or more modalities other than ultrasound images (e.g., X-ray images).

At step 206, changes in a position of the ultrasound probe and/or an orientation of the ultrasound probe over the period of time with respect to the object of interest are detected. In some embodiments, changes in a position of the object of interest and/or an orientation of the object of interest over the period of time with respect to the ultrasound probe are detected. In some embodiments, a combination of changes in the position and/or orientation of the object of interest and the ultrasound probe are detected. The detected changes can be applied to a set of analytics or predefined rules (e.g., the analytics 106 of FIG. 1) for determining a context in which the user is using the ultrasound probe. For example, rapid or large changes to the position and/or orientation of the ultrasound probe may indicate that the user is searching for the object of interest, where a broad anatomical view is advantageous. In another example, small or incremental changes to the position and/or orientation of the ultrasound probe may indicate that the user has located the object of interest and is attempting to obtain more precise or detailed images of it, where a narrower, more detailed view is advantageous. Thus, depending on the context, the ultrasound image acquisition parameters can be changed to accommodate the context, such as discussed below with respect to step 208.

At step 208, at least one ultrasound image acquisition parameter for acquiring at least one additional ultrasound image using the ultrasound probe based on the detected changes in the position and/or the orientation of the ultrasound probe is adjusted. In some embodiments, at least one ultrasound image acquisition parameter for acquiring at least one additional ultrasound image using the ultrasound probe based on the detected changes in the position and/or the orientation of the object of interest is adjusted. In some embodiments, at least one ultrasound image acquisition parameter for acquiring at least one additional ultrasound image using the ultrasound probe based on the detected changes in the position and/or the orientation of the object of interest and the ultrasound probe is adjusted. The ultrasound image acquisition parameter can include a spatial resolution of the ultrasound image, a field of view of the ultrasound image, a depth of the ultrasound signal, a frequency of the ultrasound signal, and/or an acquisition frame rate of the ultrasound image. The field of view can include a direction (e.g., in three-dimensional image acquisition, the field of view is defined by the angles or direction of multiple ultrasonic signals) and/or an angular aperture of the ultrasound signal emitted by the ultrasound probe. In one example, adjusting the field of view includes increasing (e.g., widening) the field of view or decreasing (e.g., narrowing) the field of view. In another example, adjusting the field of view includes steering or shifting the field of view such that the object of interest remains substantially encompassed within the field of view as the object of interest moves with respect to the ultrasound probe and/or as the ultrasound probe moves with respect to the object of interest. In another example, adjusting the spatial resolution includes increasing or decreasing the spatial resolution of the ultrasound image acquired by the ultrasound probe. In yet another example, adjusting the frame rate includes increasing or decreasing the rate at which frames of the ultrasound image are acquired by the ultrasound probe. In yet another example, acquisition of the ultrasound image can be automatically switched between two- and three-dimensional views and/or single or multiple scan planes.

Computer-executable instructions (e.g., the computer-executable instructions 112 of FIG. 1) may, for example, be executed by a processor (e.g., the processor 102) to perform steps 202-208 in accordance with one or more embodiments described herein.

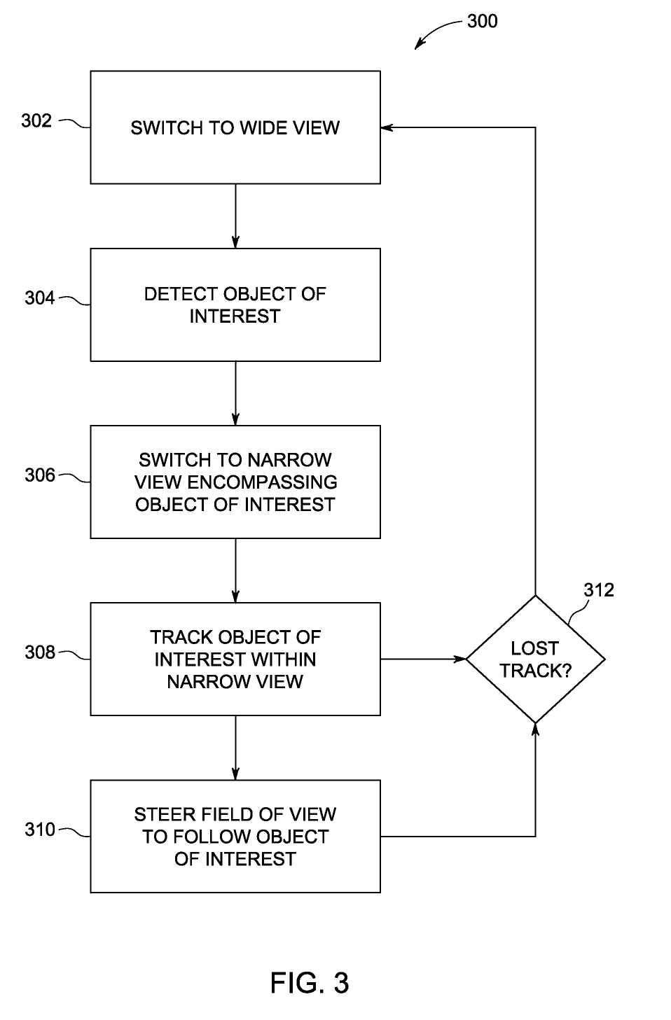

FIG. 3 is a flow diagram of an example of a process 300 for adjusting image acquisition parameters of an ultrasound image, according to an embodiment. At step 302, the field of view is switched to a wide view. As discussed above, a wide field of view may be useful when the user is attempting to locate the object of interest within a patient. For example, the wide view may provide an ultrasound image that covers a relatively large anatomical region, which may be useful while the user is attempting to locate the object of interest (e.g., surgical tool, anatomical structure, etc.). Depending on the configuration of the ultrasound probe, it may be necessary, for example, to reduce the spatial resolution of the ultrasound image and/or decrease the image acquisition frame rate while acquiring a wide field of view.

At step 304, the object of interest is identified using, for example, the object identifier 108 of FIG. 1. The object of interest may include, for example, an anatomical structure in a patient (e.g., an organ, tissue, bone, etc.), a surgical instrument inserted into the patient, a device (e.g., a valve, a pacemaker lead, a cardiac resynchronization therapy device (CRT) lead, a plug, etc.), or a marker placed into the patient. In some embodiments, a portion of a tool, such as the tip, can be the object of interest. The object of interest may, for example, be identified and detected by comparison with a statistical model (e.g., acquired from training data representing known objects) or by using other medical image analysis techniques, as will be understood by one of skill in the art.

Once the object of interest has been detected, at step 306, the field of view is automatically switched to a narrow view. For example, the narrow view may provide an ultrasound image that covers a relatively small anatomical region. As discussed above, a narrow field of view may be useful when the user is attempting to observe the object of interest in greater detail. Depending on the configuration of the ultrasound probe, it may be possible, for example, to increase the spatial resolution of the ultrasound image and/or increase the image acquisition frame rate while acquiring a narrow field of view so as to provide greater detail in the ultrasound image.

In some embodiments, at step 308, the object of interest can be tracked automatically. For example, if the object of interest and/or the ultrasound probe moves with respect to the other, the image acquisition parameters can be automatically adjusted to maintain the object of interest within the field of view. At step 310, the field of view is automatically steered or adjusted to follow certain motion of the object of interest and/or the ultrasound probe so as to maintain the object of interest within the field of view. Such steering may be obtained, for example, by adjusting the depth and/or frequency of the ultrasound probe. In some embodiments, annotations can be provided within the visualization that direct the user to manipulate the ultrasound probe in a manner that places or maintains the object of interest within the field of view. In some embodiments, the annotations direct the user to manipulate the device or tool to place or maintain the device or tool within the field of view. It will be understood, however, that beyond a certain limit of motion of the object of interest and/or the ultrasound probe (e.g., within the tolerances and capabilities of the ultrasound probe and/or the image analysis and processing algorithms), the object of interest can no longer be tracked (i.e., the tracking is lost). At step 312, if tracking of the object of interest is lost (i.e., no longer obtainable), then process 300 returns to step 302, where the field of view is automatically switched to a wide view. This enables the user to re-locate the object of interest, as described above.

Computer-executable instructions (e.g., the computer-executable instructions 112 of FIG. 1) may, for example, be executed by a processor (e.g., the processor 102) to perform steps 302-312 in accordance with one or more embodiments described herein.

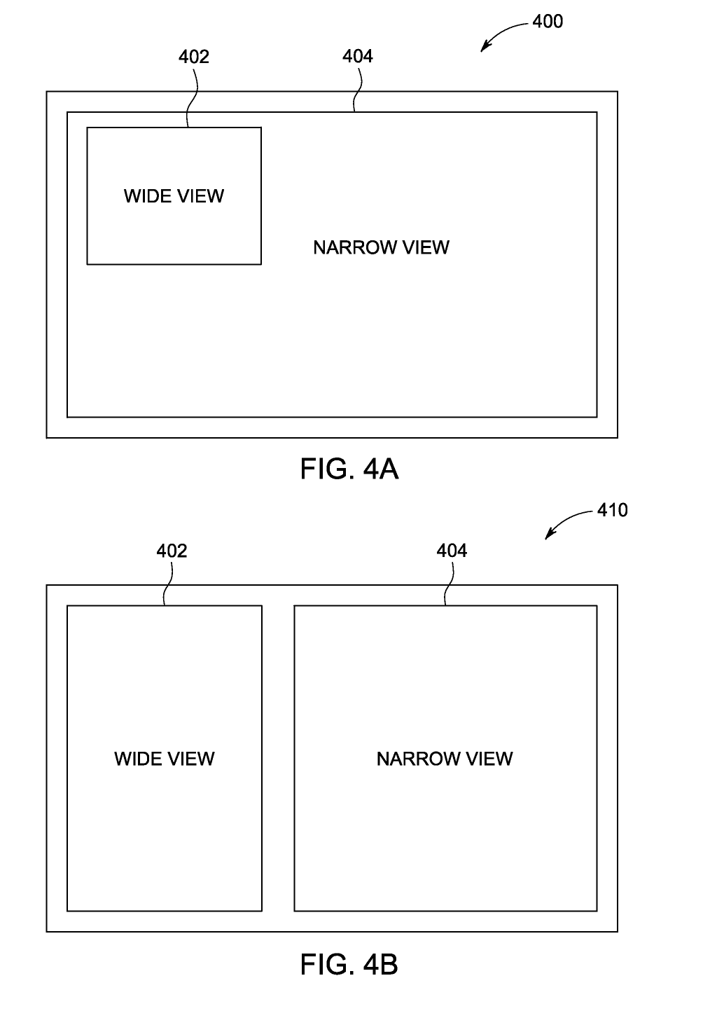

FIG. 4A depicts one example of a user interface display 400 for displaying one or more ultrasound images, according to an embodiment. In the user interface display 400, both a wide field of view 402 and a narrow field of view 404 can be displayed concurrently, with the wide field of view 402 overlaying a portion of the narrow field of view 404. In this manner both the detailed, narrow field of view 404 and the less detailed, wide field of view may be observed simultaneously in the same user interface display 400. In some embodiments, the wide field of view 402 and/or the narrow field of view 404 includes a two- or three-dimensional image. In some embodiments, the wide field of view 402 and/or the narrow field of view 404 includes a multi-planar ultrasound image.

FIG. 4B depicts another example of a user interface display 410 for displaying one or more ultrasound images, according to an embodiment. The user interface display 410 is substantially similar to the user interface display 400 of FIG. 4A, except that the wide field of view 402 and the narrow field of view 404 can be displayed side-by-side. It will be understood that the user interface displays 400, 410 are exemplary and that other configurations and arrangements of the wide and narrow fields of view 402 and 404 can be utilized in conjunction with various embodiments.

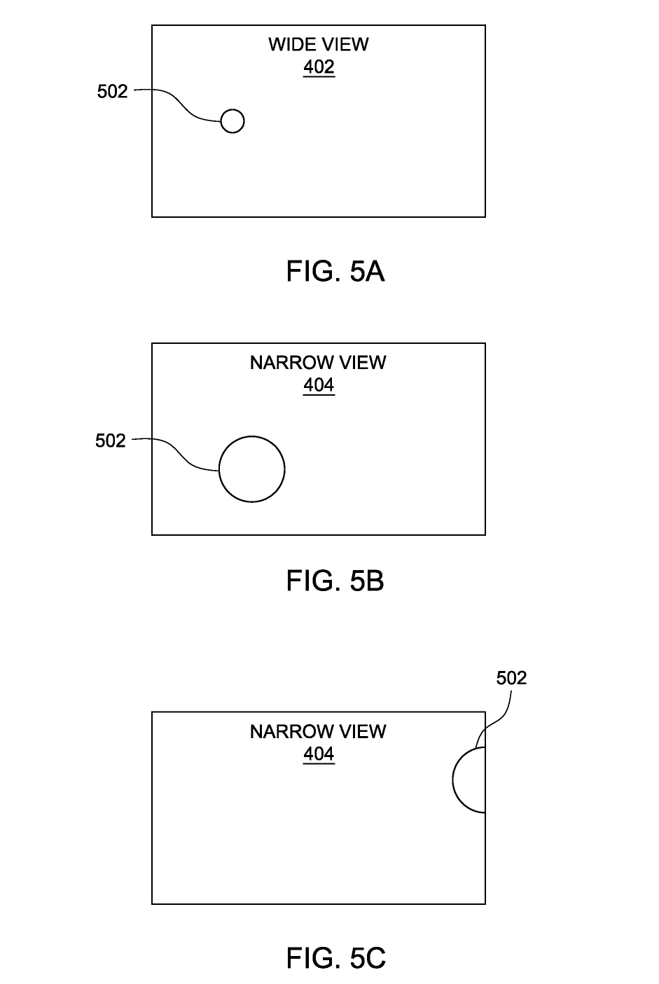

FIG. 5A depicts one example of the wide field of view 402 encompassing an object of interest 502. In some embodiments, after the object of interest 502 is detected, the field of view can automatically switch to the narrow field of view 404 such as depicted, for example, in FIG. 5B, in which the object of interest 502 can be displayed at a larger scale than in the wide field of view 402. If the object of interest 502 subsequently changes positions such that at least a portion of the object of interest 502 is no longer encompassed within the narrow field of view 404, such as depicted, for example, in FIG. 5C, the field of view can automatically switch back to the wide field of view 402 as depicted in the example of FIG. 5A.

Systems and methods disclosed herein may include one or more programmable processing units having associated therewith executable instructions held on one or more non-transitory computer readable medium, RAM, ROM, hard drive, and/or hardware. In exemplary embodiments, the hardware, firmware and/or executable code may be provided, for example, as upgrade module(s) for use in conjunction with existing infrastructure (for example, existing devices/processing units). Hardware may, for example, include components and/or logic circuitry for executing the embodiments taught herein as a computing process.

Displays and/or other feedback components may also be included, for example, for rendering a graphical user interface, according to the present disclosure. The display and/or other feedback components may be stand-alone equipment or may be included as one or more components/modules of the processing unit(s). In exemplary embodiments, the display and/or other feedback components may be used to simultaneously describe both morphological and statistical representations of a field-of-view of an ultrasound image.

The actual software code or control hardware which may be used to implement some of the present embodiments is not intended to limit the scope of such embodiments. For example, certain aspects of the embodiments described herein may be implemented in code using any suitable programming language type such as, for example, assembly code, C, C# or C++ using, for example, conventional or object-oriented programming techniques. Such code is stored or held on any type of suitable non-transitory computer-readable medium or media such as, for example, a magnetic or optical storage medium.

As used herein, a “processor,” “processing unit,” “computer” or “computer system” may be, for example, a wireless or wire line variety of a microcomputer, minicomputer, server, mainframe, laptop, personal data assistant (PDA), wireless e-mail device (for example, “BlackBerry,” “Android” or “Apple,” trade-designated devices), cellular phone, pager, processor, fax machine, scanner, or any other programmable device configured to transmit and receive data over a network. Computer systems disclosed herein may include memory for storing certain software applications used in obtaining, processing and communicating data. It can be appreciated that such memory may be internal or external to the disclosed embodiments. The memory may also include non-transitory storage medium for storing software, including a hard disk, an optical disk, floppy disk, ROM (read only memory), RAM (random access memory), PROM (programmable ROM), EEPROM (electrically erasable PROM), flash memory storage devices, or the like.

The system 100 of FIG. 1 may be any computer system, such as a workstation, desktop computer, server, laptop, handheld computer, tablet computer (e.g., the iPad® tablet computer), mobile computing or communication device (e.g., the iPhone® mobile communication device, the Android® mobile communication device, and the like), or other form of computing or telecommunications device that is capable of communication and that has sufficient processor power and memory capacity to perform the operations described herein. In exemplary embodiments, a distributed computational system may be provided including a plurality of such computing devices.

The system 100 may include one or more non-transitory computer-readable media having encoded thereon one or more computer-executable instructions or software for implementing the exemplary methods described herein. The non-transitory computer-readable media may include, but are not limited to, one or more types of hardware memory and other tangible media (for example, one or more magnetic storage disks, one or more optical disks, one or more USB flash drives), and the like. For example, the memory 104 included in the system 100 may store computer-readable and computer-executable instructions or software for implementing a graphical user interface as described herein. The processor 102, and in some embodiments, one or more additional processor(s) and associated core(s) (for example, in the case of computer systems having multiple processors/cores), are configured to execute computer-readable and computer-executable instructions or software stored in the memory 104 and other programs for controlling system hardware. Processor 102 may be a single core processor or a multiple core processor.

The memory 104 may include a computer system memory or random access memory, such as DRAM, SRAM, EDO RAM, and the like. The memory 104 may include other types of memory as well, or combinations thereof.

A user may interact with the system 100 through the display 130, which may display ultrasound images and other information in accordance with exemplary embodiments described herein. The display 130 may also display other aspects, elements and/or information or data associated with exemplary embodiments. The system 100 may include other I/O devices for receiving input from a user, for example, a keyboard or any suitable multi-point touch interface, a pointing device (e.g., a mouse, a user's finger interfacing directly with a display device, etc.). The system 100 may include other suitable conventional I/O peripherals.

The system 100 may include one or more storage devices 140, such as a durable disk storage (which may include any suitable optical or magnetic durable storage device, e.g., RAM, ROM, Flash, USB drive, or other semiconductor-based storage medium), a hard-drive, CD-ROM, or other computer readable media, for storing data and computer-readable instructions and/or software that implement exemplary embodiments as taught herein. In exemplary embodiments, the one or more storage devices 140 may provide storage for data that may be generated by the systems and methods of the present disclosure. For example, storage device 140 may provide storage for image data and/or storage for data analysis (e.g., storage for results of parameters for any of the image or statistical analyses described herein such as image segmentation results). The one or more storage devices 140 may further provide storage for computer readable instructions relating to one or more processes as described herein. The one or more storage devices 140 may be provided on the system 100 and/or provided separately or remotely from the system 100.

The system 100 may run any operating system, such as any of the versions of the Microsoft® Windows® operating systems, the different releases of the Unix and Linux operating systems, any version of the MacOS® for Macintosh computers, any embedded operating system, any real-time operating system, any open source operating system, any proprietary operating system, any operating systems for mobile computing devices, or any other operating system capable of running on the computing device and performing the operations described herein. In exemplary embodiments, the operating system may be run in native mode or emulated mode. In an exemplary embodiment, the operating system may be run on one or more cloud machine instances.

Having thus described several exemplary embodiments of the invention, it is to be appreciated various alterations, modifications, and improvements will readily occur to those skilled in the art. For example, while in some embodiments the object of interest can be identified and tracked as discussed above (e.g., using a single modality such as the ultrasound image input), in some embodiments, the object of interest may be identified and/or tracked, at least in part, using concurrent multimodal input information (e.g., ultrasound and X-ray inputs). In another example, the field of views of one or all modalities may be adjusted to optimize the tracking and acquisition of clinically useful objects of interest. Such alterations, modifications, and improvements are intended to be part of this disclosure, and are intended to be within the scope of the invention. Accordingly, the foregoing description and drawings are by way of example only.

What is claimed is:

1. A computer-implemented method for active control of ultrasound image acquisition, the computer including a processor and a memory operatively coupled to the processor, the method comprising:
accessing, by the processor, image data representing a series of ultrasound images acquired over a period of time from an ultrasound probe operatively coupled to the processor;
identifying, by the processor, an object of interest in at least one ultrasound image in the series of ultrasound images;
detecting, by the processor, at least one of:
changes in at least one of a position of the ultrasound probe and an orientation of the ultrasound probe over the period of time with respect to the object of interest; and
changes in at least one of a position of the object of interest and an orientation of the object of interest over the period of time with respect to the ultrasound probe; and
adjusting, by the processor, at least one of:
at least one ultrasound image acquisition parameter for acquiring at least one additional ultrasound image using the ultrasound probe based on the detected changes in the position and/or the orientation of the ultrasound probe; and
at least one ultrasound image acquisition parameter for acquiring at least one additional ultrasound image using the ultrasound probe based on the detected changes in the position and/or the orientation of the object of interest.

2. The computer-implemented method of claim 1, wherein the at least one ultrasound image acquisition parameter comprises at least one of:
a depth of a signal emitted by the ultrasound probe;
a frequency of the signal emitted by the ultrasound probe;
a spatial resolution of the ultrasound image;
a field of view of the ultrasound image; and
an acquisition frame rate of the ultrasound image.

3. The computer-implemented method of claim 2, wherein the step of adjusting comprises at least one of:
increasing or decreasing the spatial resolution such that the object of interest is visible within the at least one additional ultrasound image based on a set of predefined rules;
increasing or decreasing the field of view such that the object of interest appears in the at least one additional ultrasound image based on the set of predefined rules;
increasing or decreasing the acquisition frame rate based on the set of predefined rules;
increasing or decreasing the depth of the signal based on the set of predefined rules; and
increasing or decreasing the frequency of the signal based on the set of predefined rules.

4. The computer-implemented method of claim 2, wherein the step of adjusting comprises automatically steering the field of view based on the detected changes in the position and/or the orientation of the ultrasound probe and/or the object of interest such that the object of interest remains substantially encompassed within the field of view.

5. The computer-implemented method of claim 2, further comprising simultaneously displaying a wide field of view and a narrow field of view via a user interface operatively coupled to the processor.

6. The computer-implemented method of claim 1, wherein the object of interest comprises at least one of an anatomical structure in a patient, a surgical instrument inserted into the patient, a device, and a marker placed into the patient.

7. The computer-implemented method of claim 1, wherein the step of identifying the object of interest comprises using one or more image analysis techniques including:
low level feature detection;
statistical model fitting;
machine learning; and
image and model registration.

8. The computer-implemented method of claim 1, wherein at least one of the steps of identifying, detecting and adjusting are further based at least in part on concurrent multimodal input information.

9. A non-transitory computer-readable medium having stored thereon computer-executable instructions that when executed by a computer cause the computer to:
receive data representing a series of ultrasound images acquired over a period of time from an ultrasound probe operatively coupled to the processor;
identify an object of interest in at least one ultrasound image in the series of ultrasound images;
detect at least one of:
changes in at least one of a position of the ultrasound probe and an orientation of the ultrasound probe over the period of time with respect to the object of interest; and
changes in at least one of a position of the object of interest and an orientation of the object of interest over the period of time with respect to the ultrasound probe; and
adjust at least one of:
at least one ultrasound image acquisition parameter for acquiring at least one additional ultrasound image using the ultrasound probe based on the detected changes in the position and/or the orientation of the ultrasound probe; and
at least one ultrasound image acquisition parameter for acquiring at least one additional ultrasound image using the ultrasound probe based on the detected changes in the position and/or the orientation of the object of interest.

10. The computer-readable medium of claim 9, wherein the at least one ultrasound image acquisition parameter comprises at least one of:
a depth of a signal emitted by the ultrasound probe;
a frequency of the signal emitted by the ultrasound probe;
a spatial resolution of the ultrasound image,
a field of view of the ultrasound image, and
an acquisition frame rate of the ultrasound image.

11. The computer-readable medium of claim 10, further comprising computer-executable instructions that when executed by the computer cause the computer to at least one of:
increase or decrease the spatial resolution such that the object of interest is visible within the at least one additional ultrasound image based on a set of predefined rules;
increase or decrease the field of view such that the object of interest appears in the at least one additional ultrasound image based on the set of predefined rules;
increase or decrease the acquisition frame rate based on the set of predefined rules;
increase or decrease the depth of the signal based on the set of predefined rules; and
increase or decrease the frequency of the signal based on the set of predefined rules.

12. The computer-readable medium of claim 10, further comprising computer-executable instructions that when executed by the computer cause the computer to automatically steer the field of view based on the detected changes in the position and/or the orientation of the ultrasound probe and/or the object of interest such that the object of interest remains substantially encompassed within the field of view.

13. The computer-readable medium of claim 10, further comprising computer-executable instructions that when executed by the computer cause the computer to simultaneously display a wide field of view and a narrow field of view via a user interface operatively coupled to the processor.

14. The computer-readable medium of claim 9, wherein the object of interest comprises at least one of an anatomical structure in a patient, a surgical instrument inserted into the patient, a device, and a marker placed into the patient.

15. The computer-readable medium of claim 9, further comprising computer-executable instructions that when executed by the computer cause the computer to identify the object of interest by using one or more image analysis techniques including:
low level feature detection;
statistical model fitting;
machine learning; and
image and model registration.

16. A system for active control of ultrasound image acquisition, the system comprising:
a processor;
an input operatively coupled to the processor and configured to receive data representing a series of ultrasound images; and
a memory operatively coupled to the processor, the memory comprising computer-executable instructions that when executed by the processor cause the processor to:
receive data representing a series of ultrasound images acquired over a period of time from an ultrasound probe operatively coupled to the processor;
identify an object of interest in at least one ultrasound image in the series of ultrasound images;
detect at least one of:
changes in at least one of a position of the ultrasound probe and an orientation of the ultrasound probe over the period of time with respect to the object of interest; and
changes in at least one of a position of the object of interest and an orientation of the object of interest over the period of time with respect to the ultrasound probe; and
adjust at least one of:
at least one ultrasound image acquisition parameter for acquiring at least one additional ultrasound image using the ultrasound probe based on the detected changes in the position and/or the orientation of the ultrasound probe; and
at least one ultrasound image acquisition parameter for acquiring at least one additional ultrasound image using the ultrasound probe based on the detected changes in the position and/or the orientation of the object of interest.

17. The system of claim 16, wherein the at least one ultrasound image acquisition parameter comprises at least one of:
a depth of a signal emitted by the ultrasound probe;
a frequency of the signal emitted by the ultrasound probe;
a spatial resolution of the ultrasound image,
a field of view of the ultrasound image, and
an acquisition frame rate of the ultrasound image.

18. The system of claim 17, wherein the memory further comprises computer-executable instructions that when executed by the processor cause the processor to at least one of:
increase or decrease the spatial resolution such that the object of interest is visible within the at least one additional ultrasound image based on a set of predefined rules;
increase or decrease the field of view such that the object of interest appears in the at least one additional ultrasound image based on the set of predefined rules;
increase or decrease the acquisition frame rate based on the set of predefined rules;
increase or decrease the depth of the signal based on the set of predefined rules; and
increase or decrease the frequency of the signal based on the set of predefined rules.

19. The system of claim 17, wherein the memory further comprises computer-executable instructions that when executed by the processor cause the processor to automatically steer the field of view based on the detected changes in the position and/or the orientation of the ultrasound probe and/or the object of interest such that the object of interest remains substantially encompassed within the field of view.

20. The system of claim 16, wherein the memory further comprises computer-executable instructions that when executed by the computer cause the computer to simultaneously display a wide field of view and a narrow field of view via a user interface operatively coupled to the processor.

21. The system of claim 16, wherein the object of interest comprises at least one of an anatomical structure in a patient, a surgical instrument inserted into the patient, a device, and a marker placed into the patient.
